(19) United States
(12) Patent Application Publication
(10) Pub. No.: US 2005/0277578 A1
(43) Pub. Date: Dec. 15, 2005

(54) Immunity generation

(76) Inventor: Christopher Jeremy Leonard, Paignton (GB)

Correspondence Address: Melvin I. Stoltz, 51 Cherry Street, Milford, CT, 06460, US

(21) Appl. No.: 11/140,113

(22) Filed: May 27, 2005

Related U.S. Application Data
(63) Continuation-in-part of application No. 10/009,438, filed on Apr. 2, 2002, now abandoned.

(51) Int. Cl. A61K 38/16; C12P 21/06; C12N 5/06

(52) U.S. Cl. 514/2; 435/70.21; 435/348

(57) ABSTRACT

A method for the manufacture of a medicament for immunity generation includes the use of insect tissues, larval forms or derivatives of insects that have been fed on a food containing pathogens.

### RELATED APPLICATIONS

This application is a Continuation-in-Part of U.S. patent application Ser. No. 10/009,438, filed Apr. 2, 2002 entitled IMMUNITY GENERATION.

### FIELD OF THE INVENTION

This invention relates to immunity generation.

The invention also relates to the use of insects, including larval forms and other life forms in the manufacture of a medicament.

The invention further relates to a method for the manufacture of a medicament for immunity generation.

### SUMMARY OF THE INVENTION

According to a first aspect of the present invention there is provided a method for the manufacture of a medicament for immunity generation, which method includes the use of tissues, larval forms or derivatives of insects that have been fed on a food containing pathogens.

The medicament may be in a form that permits its ingestion, digestion and assimilation to provide immunity to bacterial disease. The medicament may alternatively be in a form which permits its injection into the subject to which or whom the immunity is to be given. As a further alternative, the medicament may be in a form that permits its absorption transcutaneously.

According to a second aspect of the present invention there is provided a method of generating immunity in a living creature that includes ingestion by the living creature of anti-microbial peptides.

The anti-microbial peptides are preferably provided by bacterially challenged larvae.

The living creature may be a bird or an animal, including humans.

### THE DRAWINGS

FIG. 1 is a chart showing the differential MALDI-TOF MS of hemolymphs of bacterially challenged drosphila larvae and unchallenged larvae (control),

FIG. 2 is a chart showing the differential MALDI-TOF MS of hemolymphs of fungally challenged drosphila larvae and unchallenged larvae (control),

FIG. 3 is a chart showing the differential MALDI-TOF MS of chick fed on fungally challenged drosphila larvae and blood of chick fed on unchallenged larvae (control) at end of 7^{th} day,

FIG. 4 is a chart showing the differential MALDI-TOF MS of chick fed on bacterially challenged drosphila larvae and blood of chick fed on unchallenged larvae (control) at end of 7^{th} day,

FIG. 5 is a chart showing the differential MALDI-TOF MS of chick fed on fungally challenged drosphila larvae and blood of chick fed on unchallenged larvae (control) 24 hours after last feed,

FIG. 6 is a chart showing the differential MALDI-TOF MS of chick fed on bacterially challenged drosphila larvae and blood of chick fed on unchallenged larvae (control) 24 hours after last feed,

FIG. 7 is a chart showing the differential RP-HPLC of hemolymph of fungally challenged drosophila larvae and hemolymph of unchallenged larvae 24 hours post challenge, and

FIG. 8 is a chart showing the differential RP-HPLC of hemolymph of bacterially challenged drosophila larvae and hemolymph of unchallenged larvae 24 hours post challenge.

### BEST MODE FOR CARRYING OUT THE INVENITON

The tests that have been carried out, the results of which are shown in the charts that are the subject of the accompanying drawings, provide a demonstration of the effects of transferable phylogenetically conserved bioactive molecules between species, and in particular, anti-microbial peptides.

The existence of the innate immune system has been documented for over 20 years, having first being discovered by H Boman and colleagues at the Karolinska Institute Sweden. It has now been established that the system that provides immunity for insects also provides immunity for humans. Moreover, this system is essential for humans and higher vertebrates in the first seven days of an infection, prior to the “previously thought” primary, adaptive immune system, becoming active. Without an innate immune response, a bacterial infection would overwhelm a vertebrate host before the adaptive immune system kicked in and the survival battle would already have been lost.

The activity of externally sourced anti-microbial peptides, provided intranasally, has been demonstrated for the protection of mice against influenza virus A and B.

From the initial discovery of the innate immune system and the role established for a range of inducible anti-microbial peptides, much research has been carried out to synthesise improved versions of these peptides. However, the increased antibacterial activity achieved and other pharmaceutical-modifying improvements have been to the detriment of the recipient. Researchers have not recognised the significance of AMP cytokine roles within the intra-cellular matrix nor the function of the non-active (microbial) part of the anti-microbial peptide, i.e. for the recruitment/chemo-attraction of secondary bioactive molecules, such as naive T-cells in the formulation of an integrated adaptive immune response.

The anti-microbial peptides, produced using the technology described herein, take account of evolutionary development and dietary exposure over millions of years and as such are inherently safer. Currently most antibiotics are used in isolation, resulting in selective pathogen resistance. The present invention promotes the utilisation of an array of anti-microbial peptides that work synergistically, each having a different role and function, and against which incremental gene change, within the pathogen, will not be adequate to overcome the diverse immune regulatory system that is presented to them.

Many of the original synthetically produced anti-microbial peptides were based on an all I-amino acid structure and were protease susceptible. The present invention includes the oral transfer of insect anti-microbial peptides to a recipient. The reason for the protease resistance is not yet fully understood, but it may involve the incorporation of gut-probiotic-synthesised d-amino acids, which are released as a result of bacterial degradation, transferred across the gut membrane and incorporated into peptide production machinery within the host peptides. D-amino acids are significantly more protease-resistant. The protease stability of the anti-microbial peptides may also be supported by the production of chitosan by the enzymatic breakdown of the insect chitin skeleton by host chitinase. They may also be protected by chaperone macro-globulin proteins or lipid rafts or because of post-translational modifications on the peptides that confer improved protease resistance, as well as enhanced anti-microbial activity. The ability to provide cost effective anti-microbials for our food animals is essential if they are not to become reservoirs for mutating viruses that may translocate and become human pathogens, as is the scenario with Asian chicken flu. Likewise, it is imperative that viable alternatives be found to the dwindling stock of effective antibiotics for human use.

### Experimental Method and Results

Drosophila larvae were orally bacterially challenged, with a pathogenic caratovora strain and with the entomopathogenic fungus beauveria bassiana to induce a range of antibacterial peptides. This was confirmed using MALDI-TOF MS, HPLC and Edman degradation. The larvae were fed to young chicks and their blood was subsequently analysed for the presence of the peptides and compared against control chickens that had been fed unchallenged larvae that did not contain the induced anti-microbial peptides. Maldi T of MS analysis established the presence of the drosophila anti-microbial peptides, up to 24 hours post ingestion, in chickens consuming the challenged larvae. No peptides could be detected in chickens consuming unchallenged larvae.

### Method

Chick Husbandry

Thirty-six day-old male chicks were obtained and divided equally into 3 groups, a control group to receive unchallenged drosophila larvae, a peptide-receiving group receiving bacterially challenged larvae and a third group receiving fungally challenged larvae.

The three groups were kept in separate pens, provided with water that was changed daily, and provided with a source of grit to facilitate mechanical breakdown of the larvae in the chick gut, and kept at a temperature of 25° C. Light was provided on a 12 hours on, 12 hours off basis, light commencing at 7 a.m. Chicks were provided with the appropriate fresh larvae daily at 7.30 am. Old larvae stock was removed at each new addition and at the end of the day. Chicks were provided with either unchallenged larvae (control), bacterially-challenged larvae (24 hours post challenge), or with fungally-challenged larvae—(36 hours post challenge,) as their sole source of food. In order to obtain maximum anti-microbial peptide concentration in the chicks, they were fed for a period of 7 days to best incorporate selective uptake and retention of the peptides by blood macro globulins, before being sacrificed.

Drosophila Stock

Adult drosophila were kept at 25° C. Larvae were kept at an air temperature of 29° C., though the total body mass temperature of the feeding larvae was higher. Sufficient adults were reared to provide adequate eggs and larvae to sustain the chicks over a 7-day period. The eggs were placed and the larvae raised on banana [base supply].

Bacterial Stock

A pathogenic strain of erwinia carotovora was incubated in LB medium. The bacterial strain induced antimicrobial peptide production in the larvae but did not result in larval death. There is no evidence that this bacterial strain is pathogenic to chicks or mice.

Fungal Stock

Beauveria bassiana were grown on malt-agar medium. There is evidence that this fungal strain is not pathogenic to either chicks or mice.

Bacterial Challenge

Batches of approximately 5 thousand third instar larvae raised on banana were added to a mixture of 20000 micro litres of banana that had been thoroughly mixed with 10000 micro litres of concentrated bacterial pellet. The oral challenge was undertaken in a glass vivarium with a layer of sawdust surrounding the banana mash to mop up liquid drainage and to keep the larvae centrally placed and prevent wandering.

Control larvae were taken from the original larvae stock [base supply].

Fungal Challenge

The procedure carried out was as with bacterial challenge, but substituting concentrated fungal pellet.

Some anti-microbial peptides are produced maximally 6 hours post challenge such as Drosocin trisaccharide glycoform mass 2767 daltons.

Other anti-microbial peptides reach maximum concentrations at 24 hours post challenge such as Drosocin monosaccharide glycoform mass 2401 daltons, Drosoindisaccharide glycoform mass 2564 daltons, maturated prodomain of attacin C mass 2971 daltons, Metchnikowin mass 3026 daltons, and Drosomycin mass 4889 daltons.

In order to achieve maximum AMP concentration within the insect biomass and thus increase the concentration of AMPs in the chick blood, new batches of bacterially-challenged larvae were produced on a repeat basis, providing the chicks a daily maximally AMP loaded food. To generate an array of long-term serum peptides, i.e. those detectable in hemolymph for more than two weeks post challenge, a fungal challenge was used on one group of the larvae. This induced the production of drosomycin and metchnikowin, which is known to persist for around two weeks in the hemolymph of drosophila post challenge.

Anti-Microbial Peptide Analysis

Samples of each of the bacteria and fungally challenged larvae were assayed at 6 and 24 hours post challenge for the presence of induced anti-microbial peptides and compared to unchallenged larvae, using differential MALDI-TOF MS spectra.

Larval Hemolymph Extraction

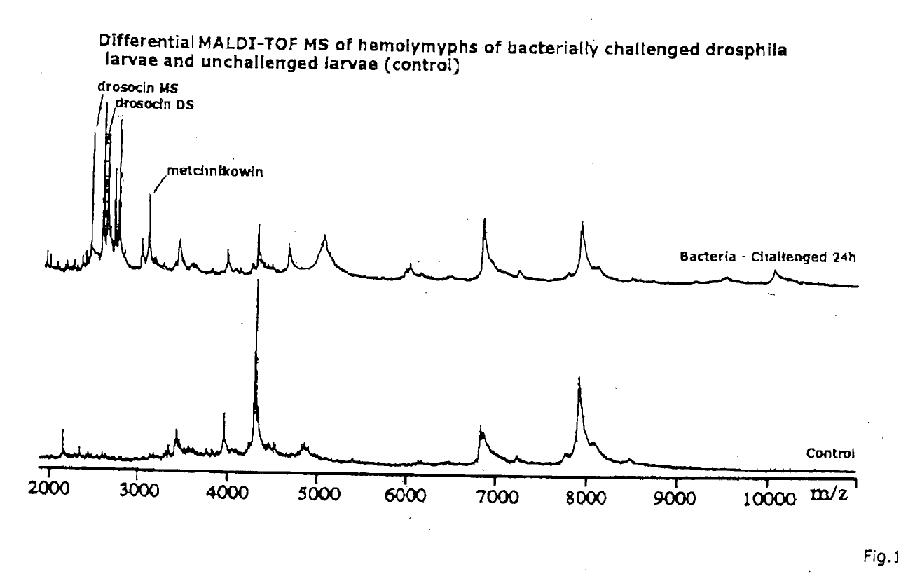

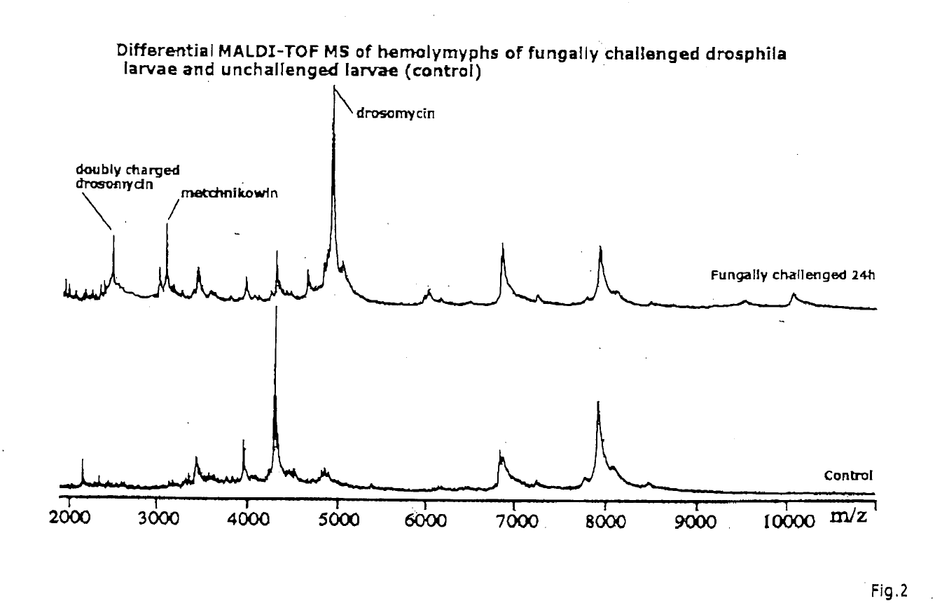

The anterior part of the larvae, bacteria challenged, fungal challenge and control were pricked and the bodies squeezed to extrude the hemolymph. Aprotinin was added to inhibit protease activity along with phenylthiourea which prevents melanization. 10 larvae were used from each of the three groups and their hemolymphs pooled for each group. The hemolymphs were then immediately centrifuged to remove blood cells [800 g, 10 minutes at 4° C. Hemolymph supernatant were subject to immediate Maldi Tof analysis. The results are shown in FIGS. 1 and 2.

### MALDI-TOF MS Analysis

The analysis was carried out on a Bruker Biflex mass spectrometer in a positive linear mode. 0.5 l of a 1:1 mixture of a —cyano-4-hydroxycinnamic acid [4 HCCA, 40 mg/ml in acetone] nitro-cellulose [NC40 mg/ml in acetone] diluted with isopropyl alcohol 1:1 was deposited on the probe tip. The hemolymph samples were loaded directly on top of the 4 HCCA/NC mixture. This was then covered by another mixture consisting of 7 mg/ml 4 HCCA in a 1:1 vol/vol with 0.10% TFA/acetonitrile. This was allowed to air dry and was then rinsed with 2 ul of 0.10% TFA and dried under vacuum.

The MALDI mass spectra of hemolymph from unchallenged larvae was externally calibrated using standard peptides angiotensin 11 [m+h]+ at m/z 1,047.2 and bovine insulin at m/z 5,734.6.

Analysis by HPLC of Drosophila Hemolymph

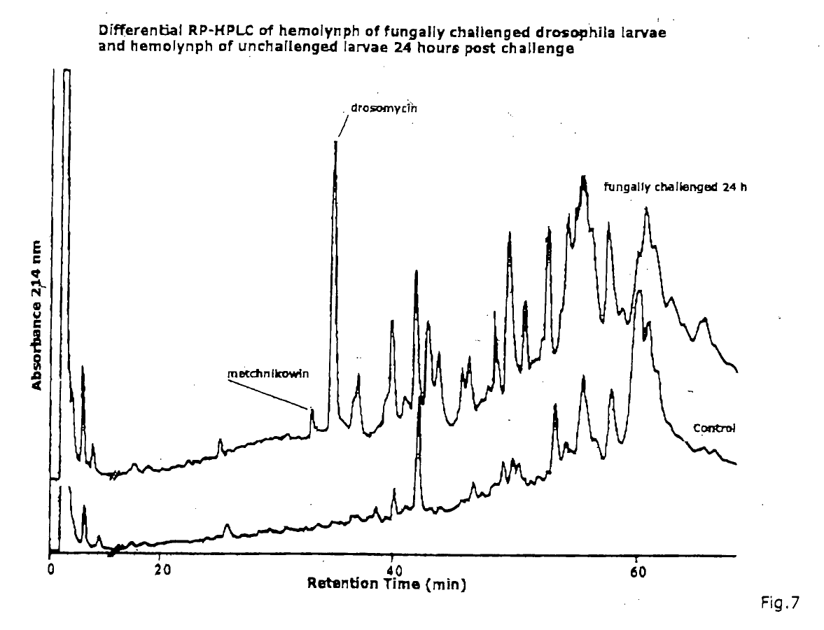

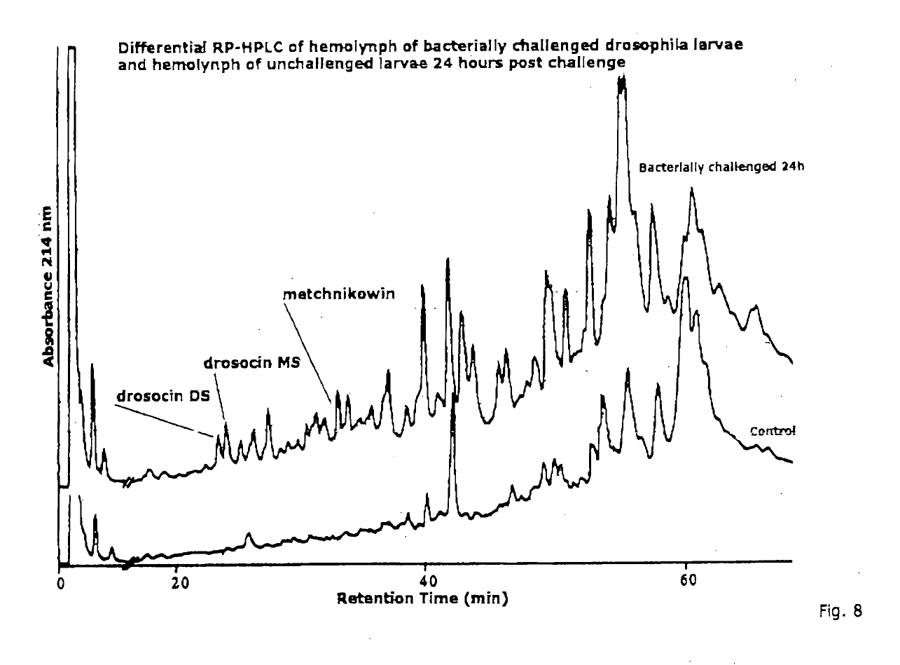

Hemolymph was collected from 50 individuals, from each of the two batches of challenged larvae [24 hours post challenge], and from the unchallenged larvae. Each sample was run independently through RP-HPLC using an aquaphore RP300 C8 column [1×100 mm] protected by a guard filter and were equilibrated in acidified water [0.05% TFA]. Separation of the hemolymph was achieved using a linear gradient of 0-85% acetonitrile in 0.05% TFA over 80 minutes with a flow rate of 80 l/min. Absorbance was monitored at 214 nm. Fractions were hand-collected into Eppendorf tubes, dried under vacuum and then reconstituted with MilliQ water then analysed with MALDI-TOF. The fractions containing drosomycin, metchnikowin and drosocin MS and drosocin DS were subject to a second purification through reverse phase chromatography to obtain pure peptides. Their structure was then confirmed using Edman degradation (see FIGS. 7 and 8).

Chick Blood Analysis

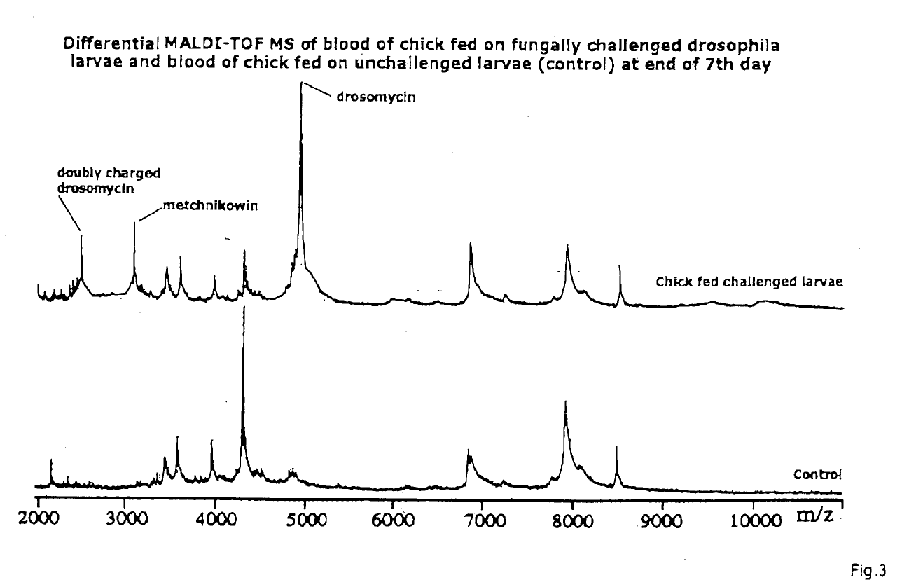

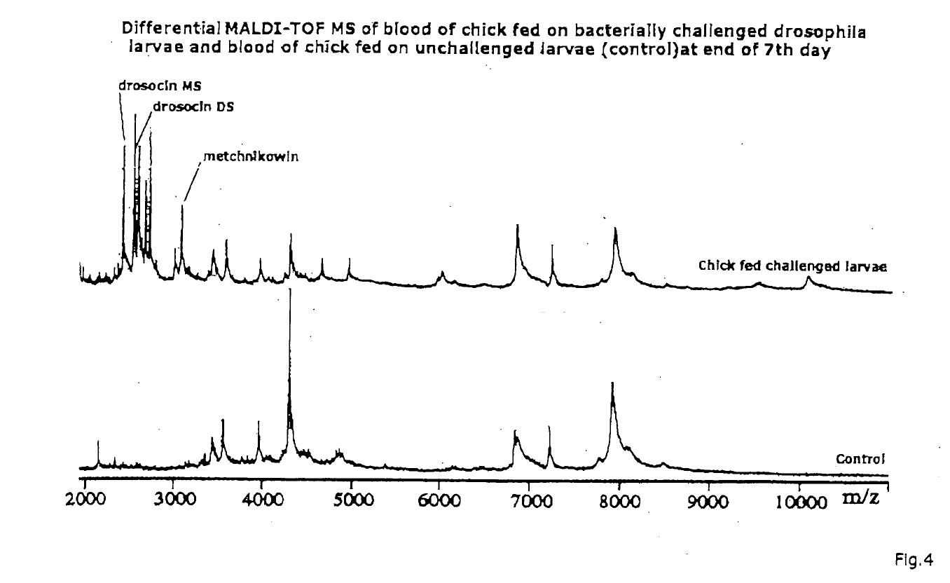

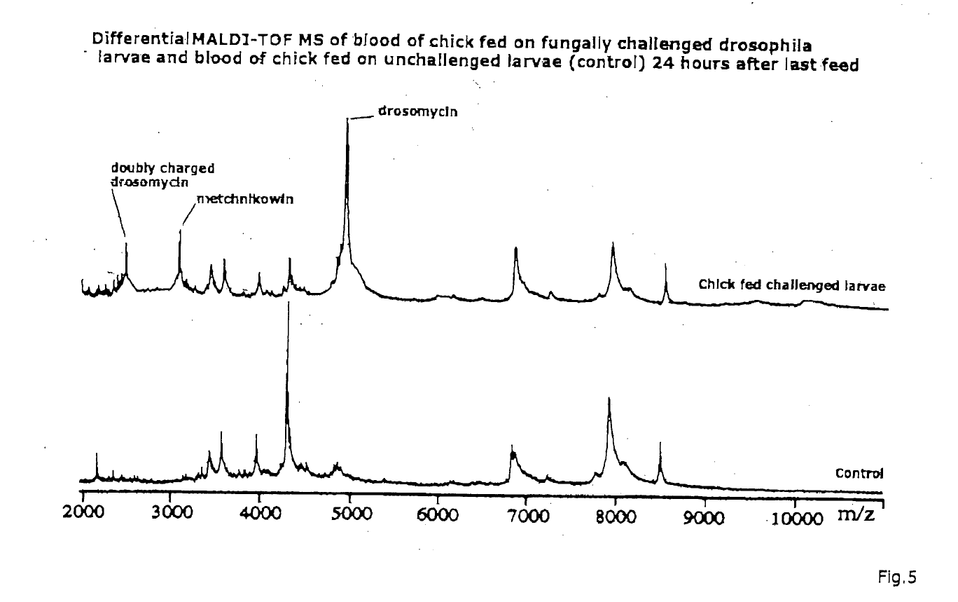

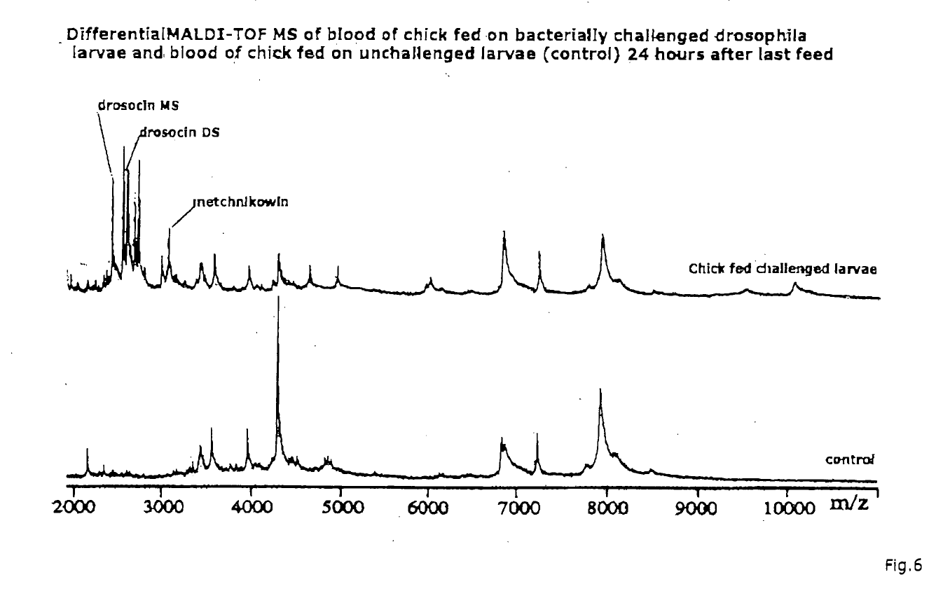

Six chicks from each of the 3 groups were sacrificed at the end of the 7th day. 2 ml blood samples were obtained from each chick and each one then individually subject to anti-microbial blood analysis HPLC/MALDI-TOF. See FIGS. 3 and 4. The remaining chicks were sacrificed at the end of the next day, having been provided with water but no larvae. Their blood samples were taken as above and made subject to HPLC, MALDO-TOF examination. See FIGS. 5 and 6.

Chick Blood Preparation

2 ml samples of blood were mixed with aprotinin and phenylthiourea and centrifuged at 3000 g for 10 minutes at 4° C. The supernatant was subject to HPLC as in the larvae analysis. MALDI-TOF spectra were obtained for specimens from the different groups and compared on a differential basis using the same standard peptides.

Confirmation of Effective Anti-Microbial Peptide Separation from Chicken Blood

In order to demonstrate that the experimental protocol to verify insect anti-microbial peptide in chicken blood post ingestion was effective, drosocin monosaccharide glycoform, metchnikowin and drosomycin that had been obtained from the chromatographic isolation of the active anti-microbial peptides in the confirmation assay from the tissues of the bacterially and fungally challenged drosophila larvae, were added to a sample of blood from a chicken that had been fed unchallenged larvae and thus did not contain any insect anti-microbial peptides. Aprotinin was added immediately to ensure no proteolytic breakdown. The spiked blood was then re-examined chroma-tographically and with maldi-tof to demonstrate the continued verifiable presence of the anti-microbial peptides and the suitability of the peptide separation techniques employed in isolating them from the larger blood serum albumen proteins.

Results

These demonstrated effective AMP production at the different stages [see FIGS. 1 to 3] and effective transfer to chick blood that could be observed 24 hours post ingestion [see FIGS. 4 to 8].

Analysis of Peptides through Edman Degradation

Structure of peptide at m/z 4,889 produced a peptide sequence:

CLSGRYKGPCAVWDNETCRRVCKEEGRSSGHCSPSLKCWCEGC43.

Peptide at m/z 2,401 produced a peptide sequence:- GKPRPYSPRPTSHPRPIRV with an O-glycosylation at threonine confirming it to be Drosocin monosaccharide.

Analysis

The research data demonstrate and confirm the viability of producing insect anti-microbial peptides by oral challenge. Moreover, peptides produced by this method were not broken down in the gut or in chicken blood nor were they excreted immediately. They could be observed in an ingestor's blood for a period of over 24 hours post feeding. A bioactive compound having these properties will be of pharmaceutical benefit to any recipient, in particular man. In view of the well-documented immune-enhancing capabilities of anti-microbial peptides in the body's defence against viral, bacterial, parasitic and tumours, these insect-based sources of antibiotics offer viable sustainable alternatives to current use antibiotics to which bacteria are becoming and have become increasingly resistant, such as methicilin-resistant staphylococcus aureus. They also offer new alternatives to current drug remedies for auto immune diseases by aiding the body in its elimination of resistant virus and suppressing TNF alpha in a feedback mechanism, reducing inflammatory cytokines, and negating the need for excess B lymphocyte production. Diseases that may be addressed using anti-microbial peptides include Rheumatoid Arthritis, Multiple Schlerosis and Diabetes.

Certain species of insects and their larvae have adapted by evolution to become biological recyclers. In this role they have developed an ability to metabolise faecal matter, dead, decaying and diseased animal remains. This has necessitated an ability on their part to withstand bacterial and viral attack from the food on which they feed, and in this way they have developed a successful immune strategy.

For the success of the animal kingdom as a whole, it is imperative that disease should not become rampant and be spread from the ingestion of the animals which feed on them.

The ingestion and digestion of insects by higher animals of the food chain provide a pathway for the transfer of immunity up the food chain.

In the move towards a more concerned approach to our environment, we need to work more closely in tandem with it. This requires a greater understanding of the way of the natural world.

In our husbandry of animals for food, we must, as far as is possible, return to a feeding regime that is more like the original regime from which the animals have developed.

It has been observed that, in the nutrition of the human species, the feeding of simple food substitutes in the form of concentrates, although providing all the basic nutritional requirements, has led to problems in the digestive tract, and a diminution of the health of the individual as exhibited by, for example, the growth in numbers of alimentary tract cancers.

What the human animal requires is good wholesome food. To ensure this, we must examine and promote the well-being of the animals on which we feed.

In the natural environment the circuit of life succeeds and we must understand and promote this relationship. In the control of disease in man, we have ventured along the path of specific antibacterial, anti-viral drug protection. In contrast, the present invention is concerned with the development of an immune system that is initiated by charging it by the ingestion of foods in which immunity has been created naturally.

### Method and Procedure

The insects to be used are grown on a substrate inoculated with the relevant bacteria or virus when pursuing specific immuniological responses. Relevant substrates would be used when biologically recycling, for example, sewage.

The insects to be used, if grown on a substrate of mixed bacteria, will provide a general raised level of anti-bacterial activity, providing general immunity to the recipient consumer. In the incidence of requiring specific immuno-response, then the particular bacteria in question will be used in the substrate. This will promote the particular lectins and induce peptides that are suitable for its control. Under the circumstances that not all of the bacteria are destroyed by the insect's immune system, the ingestion of the insect plus the residual bacteria will provide a means for the induction of the auto-immune system of the recipient consumer.

Presentation of the bacterial- and/or viral-resistant insects would be:
1 whole and live, or
2 inactivated whole, or
3 homogenised whole, or
4 sectional preparation, or
5 pupated whole live, or
6 pupated whole inactivated.

In the feeding of the insects as a food form it will be optimal to provide and present a food which does not need additional antioxidants and preservatives, as this tends to defeat the philosophy behind the concept.

The extraction of specific anti-viral and anti-bacterial activity from the insects will necessitate chemical and physical extraction processes, involving the use of stabilizers, solvents, reactants and various separation techniques.

### Insect Source

The immune response adaptability will be shown particularly amongst carrion and detritus feeders. The viability of the process is largely dependant on the ability to produce readily, large quantities and volumes of the insects of the species that are listed below. It is believed that flies, in their larval form, maggots, are the most suitable, as they have a short life span and high fecundity. Also, they have a natural inactivated inert phase—the pupa stage.

| TRUE FLIES | | SPECIES |
|---|---|---|
| | | Muscidae |
| I. | House flies | |
| | common house fly | Musca domestica |
| | common house fly | Faffia Canicularis |
| | lesser house fly | |
| | green cluster fly | Dasyphora Cyanella |
| II. | Blow flies | Calliphoridae |
| | blue bottle | Calliphora Erythrocephalia |
| | | Calliphora Vomitoria |
| | green bottle | Lucilia Sericata |
| | | Lucilia Caesar |
| | flesh fly | Sarcophaga Camaria |
| III. | seaweed fly | Coelopidae |
| | | Coelopa Fridda |
| IV. | fruit flies | Drosophilia |
| V. | crane flies | Tipulidae |
| VI. | mosquitoes | Culicidae |
| | | Andphelline |
| VII. | soldier flies | Stratiomyidae |
| VIII. | horseflies | Tabanidae |
| IX. | robber flies | Asilidae |
| X. | hover flies | Syrphidae |
| | | Tephritidae |
| XI. | louse flies | Hippoboscidae |
| | | Tachinidae |
| XII. | sewage flies | Diptera |
| 2 | cave crickets | Rhaphidophoridae |
| 3 | cockroaches | Dictyptera |
| 4 | earwigs | Dermaptera |
| 5 | ground beetles | Carabidae |
| | burying beetles | Silphidae |
| | rove beetles | Staphylinidae |
| | scarabs and chafers | Scrabaeidae |
| | click beetles | Elateridae |
| | larder beetle | Dermestidae |
| | church tard beetle | |
| 6 | centipedes | Chilopoda |
| | millipedes | Diplopodia |
| 7 | harvestmen | Opillionis |
| 8 | | Series Schizophora |
| 9 | moths and butterflies | Lepidoptera |

Superfamily Hesperioidea
Superfamily Papillonoidea
Superfamily Micropterigoidea
Superfamily Eriocranioidea
Superfamily Hepialoidea
Superfamily

Nepticuloidea/Stigmelloidea

Superfamily Incurvaroidea
Superfamily Cossoidea
Superfamily Zygaenoidea
Superfamily Pterophoroidea
Superfamily Pyraloidea
Superfamily Tortricoidea
Superfamily Sesioidea
Superfamily Tineoidea
Superfamily Alucitoidea
Superfamily Noctuoidea
Superfamily Geometroidea
Superfamily Sphingoidea
Superfamily Bombycoidea

| | |
|---|---|
| Caddis Flies | Order Trichoptera |
| Bees Wasps Ants: | Order Hymenoptera: |

Superfamily Evanioidies
Superfamily Ichneumonoidea
Superfamily Cynipoidea
Superfamily Chalcidoidea
Superfamily Proctotrupoidea
Superfamily Ceraphronoidea
Superfamily Chrysidoidea
Superfamily Scholioidea

The Ants:

Superfamily Formicoidea

The True Wasps:

Superfamily Vespoidea
Superfamily Pompiloidea
Superfamily Sphecoidea

The Bees:

Superfamily Apoidea

| | |
|---|---|
| Snails: | cochlea |
| Worms: | lumbricus terrestris |
| Slugs | |

### Diseases that could be Incorporated into this Procedure

| | |
|---|---|
| Anthrax | Bacillus Antharis |
| Botulism | Clostridium Botulinum |
| Cholera | Vibrio Cholerae |
| Diptheria | Coryne Bacterium Diptheriae |
| Food Poisoning | Staphylococcus |
| | Bacillus Cerens |
| | Clostridium Perfringens |
| | Salmonella Typhimurium |
| Gas Gangrene | Clostridium Perfringens |
| | Clostridium Novyi |
| | Clostridium Septicum |
| Gonnorrhoea | Neisseria Gonorrhoea |
| Leprosy | Mycocbacterium Leprae |
| Meningitis | Neisseria Meninigitidis |
| | Haemophilus Influenzae |
| | Streptoccus Pneumonial |
| | Listeria Monocytogenes |
| Bubonic Plague | Yersinia Pestis |
| Pneumonia | Streptococcus Pneumoniae |
| | Haemophilus Influenzae |
| | Klebsiella Pneumoniae |
| | Mycoplasm Pneumoniae |
| Q Fever | Coxiella Burneth |
| Scarlet Fever | Streptococcus Pyogenes |
| Syphilis | Treponema Palliduni |
| Tetanus | Clostridium Tetani |
| Trachoma | Chlamydia Trachomatis |
| Tuberculosis | Mycobacterium Tubercularis |
| | Mycobacterium Bovis |
| Typhoid | Salmonella Typhi |
| Typhus | Rickettsia Prowazekit |
| Measles | |
| German measles | Rubella |
| Chicken-pox | Varicella |
| Shingles | Herpes Zoster |
| Common Cold | Acute Coryza |
| Hepatitis | |
| Bee Virus | |
| Influenza | |
| Encephalitis | |
| Mumps | |
| Whooping Cough | Burdetella Pertussis |
| Small-pox | Variola |
| Rabies | Hydrophobia |

### Bacterial Families Covered by the Procedure

Escheria

Salmonella

Arizona Proteus Klebsiella Shicella

Pasteurellayersina Francisella

Bruella

Actinobacillus

Haemophilus Mora & Ella Bordatella

Spharophorus

Staphyloccus

Streptococcus

Pneumococcus

Corynebacterium

Erysipelothrix

Listeria

Bacillus

Clostridium

Mycobacterium

Actinomyces

Nocardia

Pseudomanas

Campylobacter

Leptospira

Borrelia Treponema Spirillum

Mycoplasma Achoeplasma

### Uses and Applications

Uses

A. Immunological Transfer:

1) Transfer of bacterial and viral immunity through ingestion, digestion and assimilation of insects, including their larval forms, in their whole form or processed to recipient.

2) Transfer of bacterial or viral immunity through inoculation of active extracts from the tissues of insects and their larvae to the recipient.

The procedure will also enable ‘fishy tastes’ to be eliminated, when feeding insects fed on fish waste to chickens. An important application of the present invention is thus the feeding of insects to chickens to generate innate immunity in the chickens, with the result that the eggs from the chickens are of improved quality. In addition, the innate immunity generated in the chickens can be transferred to the consumers of both the chickens and the eggs laid by the chickens.

Insects living in soil digest and assimilate plant protein including that which has been attacked by fungi. It is accordingly possible to feed the insects on specifically chosen plant protein to produce a desired innate immune response. In addition, the anti-bacterial agents in the slime of worms migrates to the skin of a worm for transfer to the soil, and hence may be absorbed by plant roots and, in this way, promote the health of the plants, providing a feedback mechanism between species members (plants) after one has succumbed to disease promoting a strengthening of the plant population (as with the animal kingdom).

The biological and digestion processes of the insect larvae will also degrade BSE proteins into their component amino aids.

The procedure will also provide a source of trace elements and minerals, including Se Selenium, Fe Iron, Mn Manganese, I Iodine, Ca Calcium, S Sulphur, K Potassium, Na Sodium, P Phosphorus and Cu Copper

Applications

1) Control of disease in man by boosting the innate immunity system and aiding the induction of the auto-immune system.

2) Control of disease in animals and fish. Thus, for poultry, protection can be obtained against avian infectious encephalomyelitis, avian listeriosis, avian plague, avian TB, avian bumblefoot, cage layer fatigue, coccidiosis, E coli, favus, fowl cholera, fowl paralysis, fowl typhoid, gapes, haemorrhagic disease, moniliasis, Newcastle disease, pullet disease, pullorum disease, salmonellosis, synovitis, toxic fat syndrome, gumboro, bronchitis, nephrosis, liver/kidney syndrome, Marek's disease and infestation with trichostrongylus axei. The invention is also applicable to the following swine diseases, i.e. dysentry, swine erysipeala, African swine fever, swine influenza, swine plague, swine pox and SVD. For cattle, protection can be obtained against actinacillosis, actinomycosis, anthrax, foot and mouth disease, brucellosis, coccidosis, pleuro-pneumonia, bovine encephalomyelitis, cattle plague, blouwildebeesoog, cerebrocortical necrosis, clostrial entiritis, johne's disease, rabies, salmonellosis, skinTB, tick-borne fever, tuberculosis, viral entiritis, virus infections of cows' teats, vulvo-vaginitismastitis, polioencephalomalacia, parasitic gastroentiritis, milk fever, red water, hypocupraemia, hypomagnesaemic disease, infectious ophthalmic disease, part-parturient haemoglobinuria, mucormycosis, mucosaldis and pyelonephritis.

3) As a food base in chickens. When given whole to chickens in a free-range environment, it provides a mode of feeding which promotes foraging and scratching. This also promotes grass and natural vegetation consumption, lessens aggressive behavior, by satisfying basic pecking response, eliminating the need for beak cutting.

4) It can also serve as a growth promoter, a means of prevention of allergies and a means for preventing or inhibiting the development and growth of tumours.

5) As a food form, it enriches the eggs that are produced from chickens. With correct use of the substrate for insects, an egg can be produced which contains Omega 3 unsaturated fatty acids, but without the fatty taste.

It can also produce an egg of lower cholesterol levels, benefitting man, the consumer.

6) As a food source for game and fowl and pheasant, grouse and partridge, particularly in their early development.

1. A method for the manufacture of a medicament for immunity generation, which method includes the use of insect tissues, larval forms or derivatives of insects that have been fed on a food containing pathogens.

2. A medicament for the provision of immunity to bacterial disease, said medicament comprising tissues, larval forms or derivatives of insects that have been fed on a food containing pathogens.

3. A medicament as claimed in claim 2, which comprises anti-microbial peptides.

4. A method of generating immunity in a living creature that includes ingestion by the living creature of anti-microbial peptides.

5. A method as claimed in claim 4, in which the anti-microbial peptides are provided by bacterially challenged larvae.
